(19) United States
(12) Patent Application Publication
(10) Pub. No.: US 2005/0158337 A1
(43) Pub. Date: Jul. 21, 2005

(54) Rehydrating personal lubricant and method of use

(76) Inventor: Jason D. Fleming, Nevada City, CA (US)

Correspondence Address: FISH & RICHARDSON P.C., PO BOX 1022, MINNEAPOLIS, MN, 55440-1022, US

(21) Appl. No.: 10/795,172

(22) Filed: Mar. 4, 2004

Related U.S. Application Data
(60) Provisional application No. 60/451,407, filed on Mar. 4, 2003.

(51) Int. Cl. A61K 35/78; A61K 31/355

(52) U.S. Cl. 424/195.18; 424/736; 424/744; 514/458

(57) ABSTRACT

A formulation is provided which acts as a rehydrating personal lubricant. A corresponding method of use is also disclosed. The formulation preferably includes guar gum and a water containing solvent. The formulation optionally but preferably additionally includes aloe vera, grapefruit seed extract, and vitamin E, as well as optionally other constituents. The formulation maintains and enhances its slipperiness over time when interacting with bodily fluids secreted by individuals utilizing the formulation during intimate contact such as sexual intercourse, such natural rehydration referred to as native rehydration.

### CROSS-REFERENCE TO RELATED APPLICATIONS

This application claims benefit under Title 35, United States Code § 19(e) of U.S. Provisional Application No. 60/451,407 filed on Mar. 4, 2003.

### FIELD OF THE INVENTION

The following invention relates to compositions and formulations having a low coefficient of friction when applied to mating surfaces, such that they function effectively as lubricants and especially as personal lubricants such as for sexual intercourse. More particularly, this invention relates to personal lubricants containing guar gum and which exhibit native rehydration when in use.

### BACKGROUND OF THE INVENTION

Intimate contact between individuals, such as during sexual intercourse, often suffers from an undesirably high amount of friction between the individuals. It is known in the prior art to provide various personal lubricants to reduce the coefficient of friction exhibited between the individuals to enhance the comfort and pleasure associated with the experience. One common category of such lubricants are lubricants formed from petroleum distillates, such as glycerine mixed with a water base. Various products are known to be provided in this category, which typically include various other ingredients in their particular formulations to distinguish them from each other. One particular such product is provided by BioFilm, Inc. of Vista, Calif. and is marketed under the trademark “ASTROGLIDE.”

ASTROGLIDE in particular includes in its formulation purified water, glycerine, propylene glycol, polyquaternium 15, methylparaben and propylparaben. Glycerine is known to be an effective lubricant and to be water soluble and generally acceptable for use directly on human skin. However, this and other petroleum distillate derived personal lubricants, such as other glycerine based lubricants, suffer from numerous drawbacks.

Specifically, such products are not organically derived, but rather manufactured through chemical engineering techniques, typically from crude oil or other petroleum products. Thus, such products suffer from all of the known, and potential unknown consequences resulting from the use of such products. Many individuals and experts have come to recognize that when products are provided which are derived from organic sources, rather than manufactured from petroleum products, that the effectiveness of such products is enhanced.

Additionally, some individuals can experience allergies or other undesirable reactions when utilizing products distilled from petroleum while not having corresponding detrimental reactions to organically derived products. Finally, many individuals recognize the benefits of the lesser impact on the environment and the benefits of sustainable develop associated with organic products. While these benefits alone suggest the desirability for the providing of personal lubricants made from materials other than petroleum distillates, such petroleum distillate lubricants also provide a less than wholly satisfactory function as a personal lubricant.

Particularly, these petroleum distillate personal lubricants experience particular negative attributes in many circumstances. Such petroleum distillate personal lubricants can experience a dehydration phenomena where, as the petroleum distillate personal lubricant formula undergoes evaporation or selective absorption such that the lubricant begins to dry out, the lubricant undergoes a radical increase in coefficient of friction, changing from slick to sticky. This phenomenon can be particularly recognized by merely placing petroleum distillate based personal lubricants on ones hands and rubbing ones hands together for about two minutes. The petroleum distillate formulation will dry out and become sticky, actually exacerbating the friction problem rather than remediating it. When water is added to the water based petroleum distillate personal lubricants they do return from sticky to slick. While this return to slickness does last for a few minutes, further dehydration causes the personal lubricant to again attain a sticky character.

Further rehydration fails to renew the petroleum distillate based personal lubricants. Rather, they attain a coefficient of friction similar to that of water when sufficient rehydration has occurred, such that the petroleum distillate based personal lubricants lose their effectiveness after about five minutes. Often such a minimal duration of function is unacceptable as the period of intimate contact has not yet ceased, resulting in discomfort or disruption for reapplication of additional lubricant.

Accordingly, a need exists for a personal lubricant which is derived from organic sources rather than from petroleum distillates. Furthermore, a need exists for personal lubricants which maintain their slipperiness for as long a duration as possible, and preferably matching the duration of the intimate contact. Such personal lubricants would particularly benefit from being readily rehydratable with liquids secreted by the individuals during intercourse, such that slipperiness is maintained for a long period of time without requiring rehydration.

### SUMMARY OF THE INVENTION

The personal lubricant of this invention is in the form of a substantially wholly organic formulation preferably including organic aloe vera juice, deionized water, organic grapefruit seed extract, vitamin E and guar gum. This preferred formulation provides a high degree of slipperiness, which is maintained for up to fifteen minutes or longer, and up to three times or more the duration of petroleum distillate based personal lubricants. This personal lubricant uniquely benefits from native rehydration. Particularly, liquids secreted by individuals, particularly during sexual intercourse, react with the personal lubricant to rehydrate the personal lubricant and extend the period during which the personal lubricant exhibits a high degree of slipperiness and a low coefficient of friction. The comfort and pleasure of the participants are thus maximized.

While the preferred formulation identified above includes all of the constituents specified, many of the benefits of this formulation can be provided with subsets of the constituents identified above. Particularly, various formulations primarily made of guar gum and water or other water containing solvents can provide many of the benefits of the formulation of this invention with a simplified formula. Furthermore, additional constituents can be added to the formulations according to this invention, including flavors, fragrances or constituents which can provide other beneficial attributes to the personal lubricant (i.e. color, aroma, medicinal value, etc.) while the personal lubricant can still maintain the enhanced slipperiness desired.

### OBJECTS OF THE INVENTION

Accordingly, a primary object of the present invention is to provide a personal lubricant which is made of all or substantially all organic constituents.

Another object of the present invention is to provide a personal lubricant which has a high degree of slipperiness.

Another object of the present invention is to provide a personal lubricant which maintains its slipperiness for a long period of time.

Another object of the present invention is to provide a personal lubricant which increases in slipperiness when rehydrated.

Another object of the present invention is to provide a personal lubricant which can be rehydrated and caused to maintain its slipperiness for a long period of time when experiencing native rehydration resulting from liquids secreted by individuals utilizing the personal lubrication during sexual intercourse.

Another object of the present invention is to provide a method for providing lubrication between individuals, such as during sexual intercourse, to minimize a coefficient of friction encountered between the individuals.

Other further objects of the present invention will become apparent from a careful reading of the included claims and detailed description of the invention.

### DESCRIPTION OF THE PREFERRED EMBODIMENT

This invention is directed to various personal lubricant formulations which exhibit a high degree of slipperiness, are made of substantially only organic substances and/or which maintain their slipperiness when rehydrated, particularly when rehydrated through the process of native rehydration. The primary constituents which make up the personal lubricant of this invention according to its preferred embodiment are guar gum and water or other water containing solvent sufficient to cause the guar gum to form a gel. The water containing solvent is preferably deionized water. The ratio of guar gum to water containing solvent is preferably approximately one to fifty. However, if the amount of the formulation provided by guar gum is between 0.01% and 15%, many of the benefits of this invention can be achieved, with formulations having an amount of guar gum between 1.0% and 10% considered to be within a most desirable range.

Guar gum is a water soluble paste made from the seeds of the guar plant. It has been known in the past as a substance useful as a thickener and stabilizer in foods and pharmaceuticals. The guar plant is an annual plant (cyamopsis tetragonolobus) native to India, adapted to semiarid regions and grown as a forage crop and for its seeds which are most often used in the production of guar gum.

Many of the benefits of the personal lubricant of this invention can be provided by merely combining the guar gum with a water containing solvent as identified above. The water containing solvent can be water alone, and preferably deionized water. This water containing solvent can also be aloe vera juice or a combination of aloe vera juice and water. This water containing solvent can also be other water containing solvents capable of converting the guar gum from a paste consistency into a gel.

The composition derives further benefits from including other constituents as follows. The composition preferably includes both water in the form of deionized water and material from the aloe vera plant, most preferably in the form of aloe vera juice. Aloe vera juice is particularly provided by filleting the leaves of the aloe vera plant and squeezing/pressing them to extract the “juice.” The aloe vera juice is preferably provided in a range between 0% and 90%. Any amount between 70% and 90% is considered most desirable, with about 78% considered to be optimal.

The preferred formulation additionally preferably includes grapefruit seed extract. This grapefruit seed extract is preferably provided as between 0% and 0.1% of the entire formulation. Most desirably the grapefruit seed extract is provided in an amount between 0.01% and 0.05% with 0.13% considered most optimal.

The preferred formulation preferably includes vitamin E as a natural preservative. The vitamin E additionally provides associated health benefits known to be associated with vitamin E. The vitamin E is preferably provided as between 0% and 5% of the entire formulation. Amounts of vitamin E between 1.0% and 3.0% are considered most desirable with 2.6% considered optimal.

Additional constituents can additionally be provided within the formulation if desired, such as flavorings, fragrances, aromas, medicinals, colorants, etc.

The process of mixing the constituents together preferably entails combining first the deionized water and organic aloe vera juice together, then adding vitamin E as a natural preservative, then adding flavor (if included), then adding grapefruit seed extract and finally adding guar gum (other constituents can be added at various different times). Everything is mixed in a food grade container. After the mix turns to gel, it is pumped into a hopper which can conveniently feed it into bottles or other sealed containers for later distribution and use.

The personal lubricant is believed to be the only lubricant on the market which uses guar gum, with the guar gum thickening the formulation into a gel and being primarily responsible for creating the slipperiness of the formulation.

Further attributes of the formulation of this invention are illustrated by reference to the following experiments. In experiment 1, a petroleum distillate based personal lubricant was applied to the hands of a test subject and the hands were rubbed vigorously while observing the passage of time. Subjective impressions of the test subject were recorded as time passed. When excessive stickiness or loss of slipperiness was encountered, rehydration was performed by adding water to the hands of the test subject. The subjective impressions of the test subject were further recorded over time as the test subject continued to vigorously rub the hands together. The experiments were continued until no appreciable enhanced lubrication was perceived.

Experiment 1

Approximately 1.0 ml of petroleum distillate personal lubricant was applied to the hands of the test subject. The petroleum distillate product involved included purified water, glycerine, propylene glycol, polyquaternium 15, methylparaben and propylparaben, and was obtained from BioFilm, Inc. of Vista, Calif. under the trademark “ASTROGLIDE.”

The subject vigorously rubbed the hands together while observing the passage of time. For the first minute the lubricant provided a very slick subjective impression. Between the end of the first minute and the end of the second minute, the subjective impression was that the lubricant was drying slightly and becoming progressively more sticky. At the two minute mark the petroleum distillate personal lubricant was uncomfortably sticky and rehydration was requested.

At this two minute mark approximately 0.5 ml of water was added to the hands of the test subject. From the two minute mark to the five minute mark the lubricant maintained a slick subjective feel. The test subject remarked that while the product remained slick the slickness was somewhat akin to the slickness provided merely by water rather than a slickness associated with the petroleum distillate product when initially applied. However, the petroleum distillate product with rehydration was considered slicker than water alone.

Between five minutes and six minutes from initiation of the testing the test subject again complained of the lubricant getting sticky and drying slightly. At the six minute mark, the test subject complained that the lubricant was uncomfortably sticky.

At the six minute mark rehydration was again performed by adding approximately 0.5 ml of water to the hands of the test subject. The test subject remarked that while some degree of slickness returned, that the slickness was akin to that of water with no appreciable added slipperiness remaining. At this point, this experiment was concluded.

Experiment 2

The same test subject thoroughly cleaned and thoroughly dried the hands. The test subject then applied approximately 1 ml of the optimal formulation of this invention to the hands and rubbed vigorously. Specifically, the formulation included 80% aloe vera juice and 20% deionized water before adding vitamin E sufficient to constitute 2.6% of the formulation, grapefruit seed extract to constitute 0.013% of the formulation and guar gum to constitute 1.3% of the formulation.

During the first minute the test subject remarked that the invention formulation provided a high degree of slickness but with progressive increase in stickiness. The test subject complained of uncomfortable stickiness after one minute from the beginning of the test.

Rehydration was performed by applying approximately 0.5 ml of water to the hands of the test subject. After rehydration, the test subject remarked that the invention formulation was slicker after rehydration than when originally applied. The slippery nature of this invention formulation after rehydration was clearly maintained until at least the three minute mark. Between the three minute mark and the four minute mark the invention formulation was identified by the test subject as getting drier with a decrease in slickness. The test subject indicated that this increase in coefficient of friction was not particularly “sticky,” but rather merely a slight loss of slickness.

At the four minute mark the test subject's hands were rehydrated with approximately 0.5 ml of water. From the four minute mark until the seven minute mark a high degree of slickness was maintained. Between the seven minute mark and the eight minute mark a progressive decrease in slickness was subjectively experienced by the test subject.

Rehydration was again performed at the eight minute mark as before. From the eight minute mark until the eleven minute mark the test subject identified the lubricant as being slick with the slickness being slightly akin to the slickness associated with merely being wet rather than the slickness associated with the lubricant. However, a significant portion of the slickness was still attributed by the test subject to the lubricant. Between the eleven minute mark and the twelve minute mark the lubricant again was perceived by the test subject as losing slickness. This loss of slickness was again identified as not being any form of “stickiness,” but rather merely a loss of slickness.

At the twelve minute mark the test subject's hands were again rehydrated as before. From the twelve minute mark to the fifteen minute mark the test subject described the lubricant as providing a slickness which was about halfway between the greatest slickness provided after initial rehydration at the one minute mark and a slickness which would be akin to a slickness provided by water alone. The test subject continued to enjoy a relatively high degree of slickness through the fifteen minute mark. Between the fifteen minute mark and the sixteen minute mark a loss of slickness was identified.

At the sixteen minute mark additional rehydration of the test subject's hands was performed as before. After this rehydration at the sixteen minute mark, the test subject identified the slickness provided as being mostly similar to a slickness derived from merely wetness, rather than a slickness associated with the invention formulation.

Based on the above experiments 1 and 2, the invention formulation exhibited nearly three times the effective duration of slipperiness (16 minutes vs. 6 minutes) when compared to personal lubricants derived from petroleum distillates. These experimental results were obtained with a test subject's hands. It is noted that human hands do not provide a significant degree of liquid secretion for rehydration, such that the provision of water from external sources was utilized. In sexual intercourse typically liquid secretion would be more prevalent such that a more consistent rehydration would occur. Additionally, while a test involving rubbing hands together occurs in an open environment where a high degree of evaporation can occur, sexual intercourse typically provides a more closed environment where less evaporation occurs.

Anecdotal evidence suggests that when the invention formulation is utilized during sexual intercourse that the invention formulation is highly effectively rehydrated with the liquids secreted by the individuals involved. Such native rehydration has been experienced which has provided a nearly unlimited duration of high degree of slipperiness with the use of the formulation of this invention. In contrast, similar use of personal lubricants derived from petroleum distillates has not been shown to be benefited particularly from any rehydration with bodily secreted liquids. Hence, this native rehydration effect, which provides significant additional duration to the effectiveness of the lubrication, has been found to be uniquely present with the formulation of this invention, and absent from prior art formulations such as those derived from petroleum distillates.

In use and operation, this formulation of this invention is preferably used as follows. The formulation is initially provided in a container which isolates the formulation from the surrounding environment. Typically, a bottle is provided from which the formulation can be squeezed in a relatively controllable fashion. The users then apply the formulation to at least one of the surfaces of one body part of one of the individuals which is to be involved in the contact between the individuals. As an acceptable alternative, the formulation can be applied to multiple body parts of one or both of the individuals. After direct application has occurred, contact between the individuals is initiated with the lubricant located between the individuals.

As an optional intermediate step, a condom or other additional device can be interposed between the individuals with the formulation either provided on the exterior surface of the condom or other device, or on the interior surface or both surfaces of the condom or other device.

After contact is initiated, such contact will typically stimulate the secretion of bodily liquids. These bodily liquids interact with the formulation in a fashion which rehydrates the formulation and enhances a slipperiness of the formulation by reducing a coefficient of friction exhibited by the formulation. Such rehydration utilizing bodily secreted liquids is referred to as native rehydration. This native rehydration continues to maintain a low coefficient of friction and a high degree of slipperiness of the personal lubricant while the contact between the individuals continues. As a result, a high degree of durational effectiveness is maintained. The individuals thus need not interrupt the activity to apply additional lubricant as frequently or require external sources of water or other rehydrating agents to maintain the slipperiness of the personal lubricant. If the individuals secrete an insufficient amount of liquid for effective native rehydration, the individuals can optionally provide water from a separate source to rehydrate the personal lubricant formulation of this invention.

When the activity ceases, the formulation is easily removed by merely washing off, as the formulation is water based.

This disclosure is provided to reveal a preferred embodiment of the invention and a best mode for practicing the invention. Having thus described the invention in this way, it should be apparent that various different modifications can be made to the preferred embodiment without departing from the scope and spirit of this disclosure.

1- A rehydrating personal lubricant, comprising in combination:
material derived from an aloe vera plant;
water;
grapefruit seed extract;
vitamin E; and
guar gum.

2- The personal lubricant of claim 1 wherein said material from an aloe vera plant includes aloe vera juice.

3- The personal lubricant of claim 1 wherein said water is deionized water.

4- The personal lubricant of claim 1 wherein said lubricant further includes a flavor.

5- The personal lubricant of claim 4 wherein said lubricant further includes a fragrance.

6- The lubricant of claim 1 wherein said guar gum is between 0.01% and 15% of the lubricant.

7- The lubricant of claim 6 wherein said guar gum is between 1.0% and 10% of the lubricant.

8- The lubricant of claim 7 wherein said guar gum is substantially 1.3% of the lubricant.

9- A lubricant for human use, comprising in combination:
up to about 15% guar gum; and
a water containing solvent.

10- The lubricant of claim 9 wherein the water containing solvent includes material from the aloe vera plant.

11- The lubricant of claim 9, wherein said lubricant further includes up to about 0.1% grapefruit seed extract.

12- The lubricant of claim 10 wherein said lubricant further includes up to about 3.0% vitamin E.

13- The lubricant of claim 9 wherein said material from the aloe vera plant includes up to about 80% aloe vera.

14- The lubricant of claim 13 wherein said aloe vera is between 70% and 90% of the lubricant.

15- The lubricant of claim 14 wherein said aloe vera is substantially 80% of the lubricant.

16- The lubricant of claim 9 wherein said guar gum is between 1.0% and 10% of the lubricant.

17- The lubricant of claim 16 wherein said guar gum is substantially 1.3% of the lubricant.

18- A method for providing lubrication between individuals, such as during sexual intercourse, including the steps of:
providing a lubricant including up to about 15% guar gum and a water containing solvent;
applying the lubricant to at least one of the individuals; and
initiating contact between the individuals with the lubricant therebetween.

19- The method of claim 18 including the further step of rehydrating the lubricant to reduce a coefficient of friction exhibited by the lubricant.

20- The method of claim 19 wherein said rehydrating step includes the step of allowing water containing liquids secreted by at least one of the individuals during said initiating step to rehydrate the lubricant.

21- The method of claim 20 wherein said providing step includes the step of including the guar gum in the amount of between 1.0% and 10% of the lubricant.

22- The method of claim 21 wherein said providing step includes the step of forming the lubricant to include substantially 1.3% guar gum, substantially 18% water, substantially 78% aloe vera, and substantially 2.6% vitamin E.
